(19) United States
(12) Patent Application Publication
(10) Pub. No.: US 2014/0127176 A1
(43) Pub. Date: May 8, 2014

(54) ORAL BACTERIA AND USES THEREOF

(71) Applicants: Linden Hu, Sharon, MA (US); Brian A. Klein, Cambridge, MA (US); Elizabeth Tenorio, Boston, MA (US)

(72) Inventors: Linden Hu, Sharon, MA (US); Brian A. Klein, Cambridge, MA (US); Elizabeth Tenorio, Boston, MA (US)

(73) Assignees: TUFTS MEDICAL CENTER, Boston, MA (US); TUFTS UNIVERSITY, Medford, MA (US)

(21) Appl. No.: 14/003,183

(22) PCT Filed: Mar. 2, 2012
(86) PCT No.: PCT/US12/27532

§ 371 Date: Dec. 16, 2013

Related U.S. Application Data
(60) Provisional application No. 61/449,394, filed on Mar. 4, 2011.

(51) Int. Cl. A61K 35/74

(52) U.S. Cl. CPC A61K 35/74
USPC 424/93.44; 424/93.4

(57) ABSTRACT

The present invention relates to compositions and methods for preventing and treating periodontitis and other conditions. In particular, the present invention relates to oral bacteria for use in treating and preventing diseases and conditions.

This application claims priority to U.S. Provisional Application No. 61/449,394, filed Mar. 4, 2011, which is herein incorporated by reference in its entirety.

### GOVERNMENT SUPPORT

This invention was made with government support under Grant R21DE016859 awarded by the National Institutes of Health. The government has certain rights in the invention.

### FIELD OF INVENTION

The present invention relates to compositions and methods for preventing and treating periodontitis and other conditions. In particular, the present invention relates to oral bacteria for use in treating and preventing diseases and conditions.

### BACKGROUND OF THE INVENTION

Periodontitis is an inflammatory condition characterized by damage to gingival tissue and periodontal ligaments, resorption of alveolar bone, and loss of teeth (Cochran, J Periodontol

2008, 79(8 Suppl):1569-76). It has also been linked to other chronic inflammatory conditions, such as diabetes, cardiovascular disease and Alzheimer's disease, leading to considerable interest in investigating the mechanisms of disease and methods for prevention (Kamer et al., Alzheimers Dement 2008, 4(4):242-50; Lamster et al., J Am Dent Assoc 2008, 139 Suppl:19S-24S; Suzuki et al., Expert Opin Ther Targets 2010, 14(10):1023-7).

Symptoms include breath odor, gums that appear bright red or red-purple, gums that appear shiny, gums that bleed easily (blood on toothbrush even with gentle brushing of the teeth), gums that are tender when touched but are painless otherwise, loose teeth and swollen gums.

The goal of treatment is to reduce inflammation, eliminate pockets if present, and address any underlying causes. Rough surfaces of teeth or dental appliances are repaired and teeth are cleaned thoroughly. This may involve use of various instruments or devices to loosen and remove deposits from the teeth (scaling). Meticulous home oral hygiene is necessary after professional tooth cleaning to limit further destruction. Surgery may be necessary. Deep pockets in the gums may need to be opened and cleaned. Loose teeth may need to be supported.

Additional therapies to treat and prevent periodontitis are needed.

### SUMMARY OF THE INVENTION

The present invention relates to compositions and methods for preventing and treating periodontitis and other conditions. In, particular, the present invention relates to oral bacteria for use in treating and preventing diseases and conditions.

Embodiments of the present invention provide compositions, kits and methods for treating or preventing periodontitis, comprising: administering a pharmaceutical composition comprising one or more (e.g., 2 or more, 5 or more or 10 or more) different bacteria (e.g., including but not limited to, one or more strains of Actinomyces naeslundii, Propionibacterium acnes, Actinomyces odontolyticus, Streptococcus intermedius, Streptococcus milleri, Streptococcus mitis, Streptococcus oralis, Streptococcus sanguinis or Veillonella dispar) to a subject exhibiting symptoms of or at risk of contracting periodontitis); wherein the administering reduces or prevents symptoms of periodontitis in the subject. In some embodiments, the bacteria is Actinomyces naeslundii 106, Actinomyces naeslundii 109A, Actinomyces naeslundii 110A, Actinomyces naeslundii 139A, Actinomyces naeslundii 141, Actinomyces naeslundii 144A, Actinomyces naeslundii 164B, Actinomyces naeslundii 181A, Propionibacterium acnes 115, Propionibacterium acnes 167, Propionibacterium acnes 168, Actinomyces odontolyticus 178, Streptococcus intermedius 173, Streptococcus milleri 165, Streptococcus mitis 166, Streptococcus mitis 185, Streptococcus oralis 180B, Streptococcus sanguinis 179 or Veillonella dispar 103 (e.g., Actinomyces naeslundii 106, Actinomyces naeslundii 139A, Actinomyces naeslundii 144A, Actinomyces naeslundii 164B, Streptococcus intermedius 173 or Streptococcus oralis 180B). In some embodiments, the administering inhibits the growth of Porphyromonas gingivalis in the subject's mouth. In some embodiments, the mode or administration is, for example, topical administration to the mouth of the subject, although the invention is not limited to a particular mode of administration. In some embodiments, the composition further comprises one or more anti-periodontitis components (e.g., fluoride, anti-inflammatory agents, analgesics, etc). In some embodiments, the pharmaceutical composition comprises a pharmaceutically acceptable carrier. In some embodiments, the pharmaceutical composition further comprises a calorie free sweetener and/or flavorings. In some embodiments, bacteria (e.g., spray dried bacteria) are added to a food or food product. Additional embodiments are described herein.

### DESCRIPTION OF THE FIGURES

FIG. 1 shows the effects of P. gingivalis (Pg) on oral epithelial cells.

A) P. gingivalis W83 (Pg), S. oralis 180B (So180B) or Pg+So180B were added to OKF6/TERT2 cells. B) OKF6/TERT2 cells were grown to confluence and used in the cell-counting assay.

FIG. 2 shows the effect of oral bacterial isolates on P. gingivalis growth. The growth of Pg was inhibited near the colonies of 6 of 19 strains including (A) S. intermedius and A. naeslundii. (B) Growth inhibition was measured as the size of the clear zone (in mm) between the edge of the colony and the lawn of P. gingivalis. Abbreviations used: An, Actinomyces naeslundii; A spp., Actinomyces spp.; Pa, Propionibacterium acnes; Si, Streptococcus intermedius; Smi, Streptococcus mitis; 5 ml, Streptococcus milleri; So, Streptococcus oralis;

Streptococcus sanguinis; Vd, Veillonella dispar. *less than 98% 16S rRNA sequence similarity to the closest type strain.

### DEFINITIONS

To facilitate an understanding of the present invention, a number of terms and phrases are defined below:

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

As used herein, the term “prokaryotes” refers to a group of organisms that usually lack a cell nucleus or any other membrane-bound organelles. In some embodiments, prokaryotes are bacteria. The term “prokaryote” includes both archaea and eubacteria.

As used herein, the term “in vitro” refers to an artificial environment and to processes or reactions that occur within an artificial environment. In vitro environments can consist of, but are not limited to, test tubes, microtiter plates, and the like. The term “in vivo” refers to the natural environment (e.g., an animal or a cell) and to processes or reactions that occur within a natural environment.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE INVENTION

The present invention relates to compositions and methods for preventing and treating periodontitis and other conditions. In particular, the present invention relates to oral bacteria for use in treating and preventing diseases and conditions.

The destruction of tissue seen in periodontitis is thought to be due to a combination of factors produced by oral bacteria and the induction of host inflammatory mediators. Although periodontitis is thought to be related to the presence of certain bacteria, no single bacterium is either necessary or sufficient to cause disease (Kuramitsu et al., Microbiol Mol Biol Rev 2007, 71(4):653-70; Nat Rev Microbiol 2010, 8(7):481-490). The present invention is not limited to a particular mechanism. Indeed, an understanding of the mechanism is not necessary to practice the present invention. Nonetheless, it is contemplated that because, in the periodontal pocket of either healthy or diseased teeth, there are many different bacteria that co-exist in close proximity to each other, that specific interactions modulate the effects on host tissue destruction. For example, one well-described interaction between different species of bacteria is in the formation of the biofilm that coats the teeth and gingival tissues, where specific, non-pathogenic bacteria are required to start the initial biofilm that allows pathogenic bacteria to attach and cause disease (Marsh et al., J Ind Microbiol 1995, 15(3):169-75; Hojo et al., J Dent Res 2009, 88(11): 982-90).

Porphyromonas gingivalis is a Gram-negative anaerobe that is one of the “redcomplex” bacteria involved in the development of periodontitis (Socransky et al., J Clin Periodontol 1998, 25(2):134-44). Porphyromonas gingivalis expresses a variety of virulence factors, including fimbriae, lectin-like adhesins, capsular polysaccharide, lipopolysaccharide, hemagglutinins, and hemolysins, and various proteolytic enzymes that cause chronic inflammation of the gingiva leading to tissue damage and loss of teeth (Malek et al., J Bact 1994, 176:1052-1059; Holt et al., Periodontol 2000, 20:168-238; Genco et al., Clin Infect Dis 1999, 28:456-465).

Experiments conducted during the course of development of embodiments of the present invention isolated and identified oral anerobic bacteria from subgingival plaque of dental patients and demonstrated that specific isolates, when co-cultured with Porphyromonas gingivalis W83, mitigated the pathogen's effects (cell rounding and wound-healing) on oral epithelial cells. Nineteen strains of Gram positive oral flora (including Actinomyces and Streptococcus oralis, S. mitis and Veilonella dispar) that protect oral epithelial cells from P. gingivalis toxicity were identified. Some of the strains were found to produce organic acids and/or hydrogen peroxide, inhibit P. gingivalis growth in plate assays, decrease the gingipain activity of P. gingivalis in co-culture or independent culture experiments, or co-aggregated with P. gingivalis cells in liquid cultures.

Accordingly, embodiments of the present invention provide compositions (e.g., pharmaceutical or research compositions or kits) comprising oral bacteria and pharmaceutical or research methods of using the bacteria in the treatment and prevention of gum and tooth disease such as periodontitis.

### I. Compositions

In some embodiments, the present invention provides compositions comprising one or more distinct isolated oral bacteria, alone or in combination with a pharmaceutically acceptable carrier or other desired delivery matiral (e.g., food prodicut, beverage, mouth wash, gum, paste, etc.). The present invention is not limited to particular oral bacteria. Examples of suitable bacteria include, but are not limited to, Actinobacteria (e.g., Actinomyces naeslundii, Actinomyces odontolyticus, Actinomyces viscosus and Propionibacterium acnes strains), Firmicutes such as Staphylococcus and Streptococcus Sp. (e.g., Bifidobacterium dentium, Gemella morbilorum, Lactobacillus rhamnosus, Staphylococcus pasteuri, Staphylococcus epidermidis, Streptococcus constellatus, Streptococcus cristatus, Streptococcus gordonni, Streptococcus intermedius, Streptococcus infantis, Streptococcus milleri, Streptococcus mitis, Streptococcus olgiofermentans, Streptococcus oralis, Streptococcus parasanguinis, Streptococcus pneumonia, Streptococcus sanguinis, Streptococcus vestibularis, Veillonella denticarosi and Veillonella dispar strains) and Fusobacteria (e.g., Fusobacterium equinum and Fusobacterium periodonticum strains). In some embodiments, the oral bacteria are one or more of A. naeslundii 106, A. naeslundii 109A, A. naeslundii 110A, A. naeslundii 139A, A. naeslundii 141, A. naeslundii 144A, A. naeslundii 164B, A. naeslundii 181A, A. odontolyticus 178, P. acnes 115, P. acnes 167, P. acnes 168, S. intermedius 173, S. milleri 165, S. mitis 166, S. mitis 185, S. oralis 180B, S. sanguinis 179 or V. dispar 103. In some preferred embodiments, the oral bacteria are one or more of A. naeslundii 106, A. naeslundii 139A, A. naeslundii 144A, A. naeslundii 164E S. intermedius 173 or S. oralis 180B.

In some embodiments, compositions comprise one or more (e.g., 2 or more, 5 or more, 10 or more, etc.) different species or strains of bacteria (e.g., selected from those described herein). In some embodiments, multiple strains of the same bacteria are utilized in combination. Any combination of 2 or 3 or 4 or 5 or 6 or 7, etc. is contemplated.

In some embodiments, the present invention provides kits, pharmaceutical compositions, or other delivery systems for use in treating or preventing periodontitis. The kit may include any and all components necessary, useful or sufficient for research or therapeutic uses including, but not limited to, one or more oral bacteria useful in preventing or treating periodontitis, pharmaceutical carriers, and additional components useful, necessary or sufficient for treating or preventing periodontitis. In some embodiments, the kits provide a sub-set of the required components, wherein it is expected that the user will supply the remaining components. In some embodiments, the kits comprise two or more separate containers wherein each container houses a subset of the components to be delivered.

Optionally, compositions and kits comprise other active components in order to achieve desired therapeutic effects.

One such desired effect can be lubrication of the oral cavity. This can be achieved by the addition of some edible oil such as Olive oil in Extra Virgin, Virgin and other cold-pressed forms, Rapeseed oil which is prepared conventionally or cold-pressed, sunflower oil, soy oil, maize oil, cotton-seed oil, peanut oil, sesame oil, cereal germ oil such as wheat germ oil, grape kernel oil, palm oil and palm kernel oil, linseed oil. Other lubricating agents, are for example essential oils such as for example eucalyptus oil, glycerin, carboxymethylcellulosa, xanthan gum or animal mucin.

In some embodiments, fluorides are added to compositions of embodiments of the present invention. In some embodiments, compositions include fluorides such as, for example, sodium fluoride, monofluorophosphate or stannous fluoride.

In some embodiments, to provide an anti-inflammatory effect, the composition of embodiments of the present invention can optionally be combined with anti-inflammatory agents such as substances like cortisone, benzydamin, non-steroid anti-inflammatory drugs or herbal extracts such as for example calendula extract or tee tree oil.

To give the composition a pleasant taste, flavoring substances such as for example mints, fruit juices, licorice, Stevia rebaudiana, steviosides or other calorie free sweeteners, rebaudioside A, essential oils like eucalyptus oil, or menthol can optionally be included in compositions of embodiments of the present invention.

In some compositions comprising oral bacteria and optionally, additional active agents, are formulated in pharmaceutical compositions. The bacteria of embodiments of the invention may be administered alone or in combination with pharmaceutically acceptable carriers or diluents, and such administration may be carried out in single or multiple doses.

The compounds of embodiments of the present invention may be isolated in any level of purity by standard methods and purification can be achieved by conventional means known to those skilled in the art, such as distillation, recrystallization and chromatography.

In some embodiments, bacteria are live cells or freeze-dried cells. Freeze-dried bacteria can be stored for several years with maintained viability. In certain applications, freeze-dried bacteria are sensitive to humidity. One way of protecting the bacterial cells is to store them in oil. The freeze dried bacterial cells can be mixed directly with a suitable oil, or alternately the bacterial cell solution can be mixed with an oil and freeze dried together, leaving the bacterial cells completely immersed in oil. Suitable oils may be edible oils such as olive oil, rapeseed oil which is prepared conventionally or cold-pressed, sunflower oil, soy oil, maize oil, cotton-seed oil, peanut oil, sesame oil, cereal germ oil such as wheat germ oil, grape kernel oil, palm oil and palm kernel oil, linseed oil. The viability of freeze-dried bacteria in oil is maintained for at least nine months. Optionally live cells can be added to one of the above oils and stored.

Compositions may, for example, be in the form of tablets, resolvable tablets, capsules, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, chewing-gums, creams, salves, jellies, gels, pastes, toothpastes, rinses, dental floss and tooth-picks, liquid aerosols, dry powder formulations, HFA aerosols or organic or inorganic acid addition salts.

The pharmaceutical compositions of embodiments of the invention may be in a form suitable for oral, topical, buccal administration. Depending upon the disorder and patient to be treated and the route of administration, the compositions may be administered at varying doses.

For oral or buccal administration, the oral bacteria of embodiments of the present invention may be combined with various excipients. Solid pharmaceutical preparations for oral administration often include binding agents (for example syrups, acacia, gelatin, tragacanth, polyvinylpyrrolidone, sodium lauryl sulphate, pregelatinized maize starch, hydroxypropyl methylcellulose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, calcium phosphate, glycine magnesium stearate, starch, rice flour, chalk, gelatin, microcrystalline cellulose, calcium sulphate, and lactitol). Such preparations may also include preservative agents and anti-oxidants.

Liquid compositions for oral administration may be in the form of, for example, emulsions, syrups, or elixirs, or may be presented as a dry product for reconstitution with water or other suitable vehicle before use. Such liquid compositions may contain conventional additives such as suspending agents (e.g. syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavouring, preservative, sweetening or colouring agents. Diluents such as water, ethanol, propylene glycol, glycerin and combinations thereof may also be included.

Other suitable fillers, binders, disintegrants, lubricants and additional excipients are well known to a person skilled in the art.

In some embodiments, two bacteria are spray-dried. In other embodiments, bacteria re suspended in an oil phase and are encased by at least one protective layer, which is water-soluble (water-soluble derivatives of cellulose or starch, gums or pectins; See e.g., EP 0 180 743, herein incorporated by reference in its entirety).

In some embodiments, bacteria (e.g., spray-dried bacteria) are added to a food or food product, gum, etc (See e.g., EP 0 862 863 and EP 0 704 016, each of which is herein incorporated by reference in its entirety). For example, in some embodiments, spray-dried bacteria are mixed into a carrier substrate, which may be water, fat or a protein digest, and the mixture is then sprayed onto or mixed into a food product.

### II. Uses

The compositions of embodiments of the present invention comprising one or more oral bacteria find use in a variety of research and therapeutic applications. In some embodiments, compositions are used in research applications (e.g., to study the mechanisms of periodontitis and other oral diseases and in the development of new therapies for periodontitis).

In some embodiments, compositions (e.g., pharmaceutical compositions) of embodiments of the present invention find use in therapeutic applications. In some embodiments, therapeutic uses comprise treating and preventing oral or gum disease (e.g., periodontitis). In some embodiments, pharmaceutical compositions are administered to a subject who does not have periodontitis, in order to prevent development of symptoms of periodontitis. In some embodiments, the subject is at risk of developing periodontitis.

In other embodiments, pharmaceutical compositions are administered to subjects exhibiting symptoms of periodontitis or related disorders in order to treat or reduce symptoms of disease. In some embodiments, the administering prevents progression of disease (e.g., prevents further gum damage). In some embodiments, the administering reverses symptoms or existing damage.

In some embodiments, pharmaceutical compositions are administering in a maintenance or ongoing manner (e.g., one or more times a day, two or more times a day, one or more times a week, etc.). In some embodiments, compositions are administered continuously (e.g., via a dental apparatus or time release formulation). In some embodiments, compositions are administered once, twice, 5 times, 10 times or more. In some embodiments, compositions are administered over a period of weeks, months, years or indefinitely.

Additional uses are within the scope of one of skill in the art.

### EXPERIMENTAL

The following examples are provided in order to demonstrate and further illustrate certain preferred embodiments and aspects of the present invention and are not to be construed as limiting the scope thereof.

### Example 1

### A. Methods

### Bacterial Strains and Growth Conditions

Porphyromonas gingivalis strain W83 (ATTC BAA-308) was cultured anaerobically in Blood Agar containing Tryptic Soy (TS) with 5% sheep blood, or Brain Heart Infusion (BHI) broth supplemented with Yeast Extract, hemin (10 μg/ml, Vitamin K (1 μg/ml) and cysteine according to standard protocols (Grenier, J Clin Microbiol 1996, 34(5):1249-52). P. gingivalis was incubated for 2-3 days in broth media at 37° C. inside anaerobic gas pouches (BD GasPak Easy). Bacterial cells were collected by centrifugation and suspended to OD₆₀₀=1 in Phosphate-buffered saline (PBS).

Clinical oral strains were isolated on Blood Agar from subgingival plaque samples collected during routine cleaning from patients being treated at the Tufts University Dental Medicine Clinic. The 16S rRNA sequences of the clinical isolates amplified using 16S rRNA Universal Primers (Song et al., Anaer 2004, 10:179-184) were compared to microbial genome databases using BLAST2TREE. The most closely related species was identified by percent sequence similarity.

### Host Cell Culture

The immortalized oral epithelial cell line OKF6/TERT-2 was cultured in Keratinocyte-serum free medium supplemented with epidermal growth factor and bovine pituitary extract (Gibco) as described (Dickson et al., Mol Cell Biol 2000, 20(4):1436-47). Primary human gingival epithelial cells (HGEC) were purchased from CELLnTEC Advanced Cell Systems cultured in PCT (Progenitor Cell Targeted) Oral Epithelium Medium according to the manufacturer's instructions.

### Bacterial and Cellular Co-Cultures

The effect of clinical isolates on the cytotoxicity of P. gingivalis was determined by measuring the ability of mixed bacterial cell suspensions to cause morphological changes in oral epithelial cells as previously described (Shah et al., J Periodontol 1992, 63(1):44-51; Morioka et al., J Periodontol 2004, 75(6):858-65; Furuta et al., Infect Immu 2009, 77(11): 4761-70). Monolayers of the OKF6/TERT-2 grown in 48-well tissue culture plates were wounded by scratching with a pipet tip. P. gingivalis cells (M01=100) with or without another species of bacteria (M01=100) were added to the cell culture. The co-cultures were incubated overnight at 37° C. in a humidified atmosphere with 5% CO₂. Uninfected host cells or cells infected with P. gingivalis alone were used as the negative and positive controls, respectively. The epithelial cells were observed for morphological changes such as cell rounding, detachment and wound healing using an inverted phase-contrast microscope at 200× magnification (Nikon TMS) and graded “−”, “+” or “++” (extent of cell rounding and detachment) by blinded observers. The assays were repeated for HGEC cells using the same protocol.

### Assay for Inhibition of Gingipain Activity

Determination of the effects of microbial interactions on P. gingivalis cytotoxicity related to gingipain activity was performed using a modification of the strategy described in Bakri & Douglas (Bakri et al., Arch Oral Biol 2005, 50(7):645-51). Briefly, strains to be tested were anaerobically grown in TS broth for 2 days. Ten μl of broth cultures were mixed with 10 μl of P. gingivalis culture and 80 μl of the Arg-gingipain substrate benzoyl-arginine naphthylamide (BANA, 440 μg/ml) in 100 mM Tris-HCl buffer with 2 mm dithiothreitol and incubated at 37° C. for 2 h. Five microliters of Fast BB solution (350 μg/ml in ethylene glycol, MP Biomedicals) was added and the OD490 nm was measured after 30 min using a spectrophotometer. Assays were performed in duplicates and confirmed a minimum of two times.

Assays for Inhibition of P. gingivalis Growth

Determination of the capacity for isolated strains of bacteria to inhibit growth of P. gingivalis by agar plate assay (Grenier, supra) were performed by first plating 100 μl of P. gingivalis overnight BHI broth culture onto a Blood Agar plate. After drying, 10 μl of overnight broth cultures of the individual clinical isolates were spotted on the plate. Inhibitory activity was determined by measuring the clear zone between the tested strains and P. gingivalis. Inhibition experiments were also performed using UV-killed cells (resuspended in PBS and spotted on the P. gingivalis lawn) and cell-free supernatants (10 ul aliquots deposited onto round wells cut into the agar plate and allowed to diffuse before inoculation with P. gingivalis).

### Assays for Acid Production

Identification of strains that produce organic acid metabolites was performed by inoculation of overnight cultures of the clinical isolates into TS broth and plates containing phenol red as pH indicator. Cultures were incubated anaerobically at 37° C. for 2 days. Cultures were monitored visually for a shift in color indicating a pH shift from 7.0 to less than 6.8. The pH of broth cultures was measured using Hydrion pH papers (range of 3.5 to 8.0, Microlab Essentials).

### Assay for Production of Hydrogen Peroxide (H₂O₂)

Testing for H₂O₂ production was performed by identification of alpha-hemolysis of red blood cells (greenish discoloration of the area surrounding the colony) on Blood Agar plates after incubation for 2 days at 37° C. To determine if the strains actively produced H₂O₂ in the presence of P. gingivalis, 100 μA of an overnight culture of P. gingivalis was spread onto the surface of Blood Agar plates; after drying, 10 μl of overnight broth cultures of inhibitory strains were spotted on the plates and likewise observed for alpha-hemolysis. To test whether H₂O₂ production is differentially regulated in liquid media, a colorimetric assay modified for liquid cultures was performed (Doran et al., Microb Ecol Health Dis 2004, 16:23-27). Ten μl TS broth cultures of each inhibitory strain was mixed with 90 μl TMB substrate (SureBlue, KPL Protein Research Products) and 1:200 dilution of horseradish peroxidase (R&D Systems), incubated at room temperature and observed for the development of blue color. Medium alone was used as negative control while medium with 0.3% H₂O₂ was used as positive control. Results of duplicates from three independent assays were reported.

### Assay for Aggregation

The ability of the inhibitory strains to co-aggregate with P. gingivalis was assessed as described (Marcotte et al., J App Microbiol 2006, 100:256-263). Briefly, bacteria were cultured in TS broth to stationary phase then 30 μl of P. gingivalis cell suspension was added to 30 μl of inhibitory strain suspension in 96-well microtiter plates. The plates were gently shaken and aggregation was scored visually under a light microscope and confirmed by Gram staining. Autoaggregation of pure cultures was likewise observed. Results of triplicates from three independent assays were reported.

### B. Results

### Isolation of Oral Bacteria

To obtain bacterial isolates for studies, patients attending the Tufts School of Dental Medicine Periodontology clinic were recruited. A total of 103 strict and facultative anaerobic strains representing 26 different species were isolated from dental plaque (Table 1). The majority of the isolates were Gram-positive species. Streptococcus and Actinomyces are the predominant genera isolated in this study.

Effects of Clinical Isolates on Cytotoxicity of P. gingivalis In Vitro

To determine whether oral flora from subjects with periodontitis may interact with pathogenic bacteria known to cause periodontitis, two in vitro models of cytotoxicity with P. gingivalis were utilized. First, a cellular cytotoxicity assay (Shah et al., supra; Morioka et al, supra) where P. gingivalis W83 is added to confluent cell cultures resulting in morphological changes in both the oral epithelial cell line OKF6/TERT-2 and in primary human gingival epithelial cells (HGEC) was utilized. These changes included loss of adhesion, cell rounding and detachment. Next, a wound-healing model of P. gingivalis cytotoxicity (Furuta et al., supra) where confluent cell cultures of OKF6/TERT-2 or HGEC were injured by creating a linear streak was utilized. Wounded cells that were not exposed to bacteria repaired the gap and fully recovered within 12 hrs. In contrast, cells treated with P. gingivalis showed greatly impaired ability to grow and repair the wound.

To determine whether the presence of other bacteria could affect cytotoxicity induced by P. gingivalis, individual clinical isolates were added to co-cultures of P. gingivalis and cells. Differences in cytotoxicity were observed upon the coinfections of host cells with combinations of P. gingivalis and some clinical isolates (Table 1). Results are shown for the cytotoxicity assay. Results for the wound healing model were in agreement with the cytotoxicity assay. Nineteen clinical isolates that weakly or strongly reduced the cytotoxic effects of P. gingivalis were identified and selected for further study. Not all strains from a specific species of bacteria showed similar effects on P. gingivalis cytotoxicity. Not all bacteria grew equally well under these conditions so that, although equivalent numbers of bacteria were added at the beginning of the assay, the numbers were often different by the end. P. gingivalis, which is an anaerobic bacterium, survives but did not show growth under these conditions (as determined by plating of the co-cultures on blood agar plates after incubation). The present invention is not limited to a particular mechanism. Indeed, an understanding of the mechanism is not necessary to practice the present invention. Nonetheless, there did not appear to be a correlation between growth of the commensal bacteria and inhibition of P. gingivalis cytotoxicity, indicating that inhibition cannot be explained solely on the basis of competition for nutrients.

Effects of Clinical Isolates on Gingipain Activity from P. gingivalis

An investigation of how the clinical isolates affect P. gingivalis cytotoxicity was next performed. Host cell detachment, cell adhesion molecule cleavage and apoptosis have been attributed to gingipains, a major class of virulence factors produced by P. gingivalis (Sheets et al., Infect Immun. 2006, 74(10):5667-5678; Stathopoulou et al., BMC Microbiol 2009, 9:107). Gingipains are trypsin-like cysteine proteases linked to the destruction of periodontal tissues, alveolar bone loss and as modulation of the host immune system (Pathirana et al., Infect Immun 2007, 75(3):1436-42). The 19 strains of inhibitory bacteria identified above were co-cultured with P. gingivalis W83 and measured Arginine-gingipain activity using the BANA test. It was found that 5 of 19 strains inhibited gingipain activity by greater than 25% when co-cultured simultaneously with P. gingivalis (Table 2). Six strains showed inhibition of gingipain activity when cultured separately from P. gingivalis and mixed and analyzed immediately.

Effects of Clinical Isolates on Growth of P. gingivalis

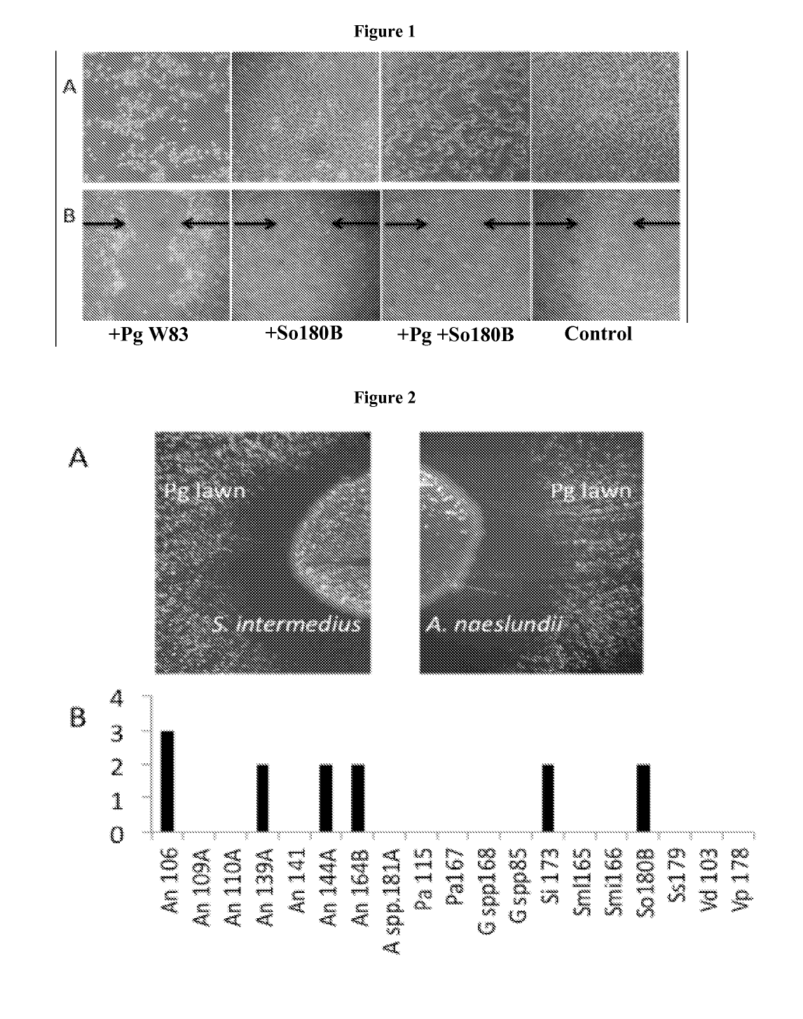

Another potential mechanism by which interactions between strains may affect the cytotoxicity caused by P. gingivalis is through affecting replication of the organism. This can affect cytotoxicity either by affecting gingipain production or through effects on other virulence factors. The ability of the strains that mitigated the cytotoxicity of P. gingivalis to affect growth was investigated by agar plate assay (Grenier, supra). Four A. naeslundii isolates, one S. intermedius and one S. oralis inhibited growth of P. gingivalis as determined by a zone of clearing when grown on a lawn of P. gingivalis on a Blood Agar plate (FIG. 1). The inhibitory properties of these strains required active cell growth since cell-free supernatants or UV-killed cells did not affect P. gingivalis growth.

### Acid and H₂O₂ Production by Clinical Isolates

Production of acid and H₂O₂ are among the mechanisms used by some oral bacteria to control the growth of other bacteria (Doran et al, supra; Hillman et al., Arch Oral Biol 1985, 30(11-12):791-5′; Tong et al., Mol Microbiol 2007, 63(3):872-80). The acid and H₂O₂ producing ability of the inhibitory strains of bacteria were determined (Table 2). Thirteen strains were shown to be acid producers in liquid cultures while 3 strains were H₂O₂ producers in plated cultures. All 8 A. naeslundii isolates tested positive for acid production while only 2 were positive for H₂O₂. Among the 5 Streptococcus species studied, only S. intermedius 173 and S. oralis 180B lowered the pH of the cultures to acidic range. There was a relationship between the ability to produce acid or H₂O₂ and the ability to inhibit growth of P. gingivalis.

### Aggregative Properties of Clinical Isolates

Adhesion of various strains to themselves or to other bacteria can affect the presentation of bacteria to cells and components of the immune system through the formation of biofilms or clusters that sequester bacteria. Using a microplate assay, auto-aggregation in pure cultures of Veillonella dispar 103, and four strains of A. naeslundii (139A, 141, 144A and 164B) was observed. When co-cultured with P. gingivalis, co-aggregation was observed with each of the 5 strains as determined by gram staining. Porphyromonas gingivalis cells were observed within the cell clusters of the autoaggregative strains under the microscope at 1000× magnification. P. gingivalis did not cluster any of the non-auto-aggregative strains.

TABLE 1
Inhibition of P. gingivalis cytotoxicity by clinical strains

| | | Inhibition of | | |
|---|---|---|---|---|
| | No. of | PG cytotoxicity | | |
| Classification of clinical isolates | strains | − | + | ++ |
| Actinobacteria (Gram+, high G + C ratio) | | | | |
| Actinomyces naeslundii | 13 | 5 | 4 | 4 |
| Actinomyces odontolyticus | 2 | 1 | 0 | 1 |
| Actinomyces viscosus | 1 | 1 | 0 | 0 |
| Propionibacterium acnes | 3 | 0 | 2 | 1 |
| Firmicutes (Gram+, low G + C ratio) | | | | |
| Bifidobacterium dentium | 1 | 1 | 0 | 0 |
| Gemella morbilorum | 1 | 1 | 0 | 0 |
| Lactobacillus rhamnosus | 2 | 2 | 0 | 0 |
| Staphylococcus pasteuri | 2 | 2 | 0 | 0 |
| Staphylococcus epidermidis | 2 | 2 | 0 | 0 |
| Streptococcus constellatus | 1 | 1 | 0 | 0 |
| Streptococcus cristatus | 1 | 1 | 0 | 0 |
| Streptococcus gordonni | 2 | 2 | 0 | 0 |
| Streptococcus intermedius | 3 | 2 | 1 | 0 |
| Streptococcus infantis | 2 | 2 | 0 | 0 |
| Streptococcus milleri | 2 | 1 | 0 | 1 |
| Streptococcus mitis | 5 | 3 | 0 | 2 |
| Streptococcus olgiofermentans | 1 | 1 | 0 | 0 |
| Streptococcus oralis | 13 | 12 | 0 | 1 |
| Streptococcus parasanguinis | 2 | 2 | 0 | 0 |
| Streptococcus pneumoniae | 3 | 3 | 0 | 0 |
| Streptococcus sanguinis | 17 | 16 | 0 | 1 |
| Streptococcus vestibularis | 3 | 3 | 0 | 0 |
| Veillonella denticarosi | 2 | 2 | 0 | 0 |
| Veillonella dispar | 2 | 1 | 0 | 1 |
| Fusobacteria (Gram−) | | | | |
| Fusobacterium equinum | 1 | 1 | 0 | 0 |
| Fusobacterium periodonticum | 1 | 1 | 0 | 0 |
| Unidentified oral isolates | 15 | 15 | 0 | 0 |
| Total | 103 | 84 | 7 | 12 |

TABLE 2
Inhibition of P. gingivalis gingipain activity and production of acid and
hydrogen peroxide by oral bacterial strain

| | Percent inhibition of gingipain | | Production of | |
|---|---|---|---|---|
| | Co-cultured | Independent | Acid | |
| Clinical isolates | with PG | Culture | (pH) | H₂O₂ |
| A. naeslundii 106 | — | 23% | + (6.0) | − |
| A. naeslundii 109A | 16% | 15% | + (5.8) | − |
| A. naeslundii* 110A | — | 26% | + (5.8) | + |
| A. naeslundii 139A | 3% | — | + (5.8) | − |
| A. naeslundii 141 | — | 10% | + (5.8) | − |
| A. naeslundii 144A | — | — | + (5.8) | − |
| A. naeslundii* 164B | — | — | + (5.8) | − |
| A. naeslundii* 181A | — | 37% | + (6.4) | + |
| A. odontolyticus* 178 | — | 11% | − (7.0) | − |
| P. acnes 115 | — | — | − (7.0) | − |
| P. acnes 167 | 3% | 41% | + (5.8) | − |
| P. acnes 168 | 26% | 8% | − (7.0) | − |
| S. intermedius 173 | — | — | + (5.5) | − |
| S. milleri 165 | 34% | — | − (7.0) | − |
| S. mitis 166 | 43% | 37% | + (6.2) | − |
| S. mitis* 185 | — | 35% | − (7.0) | − |
| S. oralis 180B | — | 28% | + (6.2) | + |
| S. sanguinis 179 | 60% | 17% | + (6.6) | − |
| V. dispar 103 | 27% | 5% | + (6.2) | − |

Table 3 shows GenBank accession numbers of the rRNA sequences of the strains described herein. All samples were isolated from subgingival plaque samples using blood agar isolation media and grown under anaerobic conditions.

TABLE 3

| GenBank | | | | |
|---|---|---|---|---|
| Accession | | | Strain | |
| Number | Banklt Number | Identifier | No. | Strain Name |
| JF496718 | Banklt1436913-1 | Seq106_P3 | 106 | A. naeslundii |
| | | | | 106 |
| JF496719 | Banklt1436913-2 | Seq109_P3 | 109A | A. naeslundii |
| | | | | 109A |
| JF496720 | Banklt1436913-3 | Seq110A_P3 | 110A | A. naeslundii* |
| | | | | 110A |
| JF496721 | Banklt1436913-4 | Seq139A_P3 | 139A | A. naeslundii |
| | | | | 139A |
| JF496722 | Banklt1436913-5 | Seq141A_P3 | 141 | A. naeslundii |
| | | | | 141 |
| JF496723 | Banklt1436913-6 | Seq144A_P3 | 144A | A. naeslundii |
| | | | | 144A |
| JF496724 | Banklt1436913-7 | Seq164B_P3 | 164B | A. naeslundii* |
| | | | | 164B |
| JF496725 | Banklt1436913-8 | Seq181A_P3 | 181A | A. naeslundii* |
| | | | | 181A |
| JF496726 | Banklt1436913-9 | Seq178_P3 | 178 | A. |
| | | | | odontolyticus* |
| | | | | 178 |
| JF496727 | Banklt1436913-10 | Seq115_P3 | 115 | P. acnes 115 |
| JF496728 | Banklt1436913-11 | Seq167_P3 | 167 | P. acnes 167 |
| JF496729 | Banklt1436913-12 | Seq168_P3 | 168 | P. acnes 168 |
| JF496730 | Banklt1436913-13 | Seq173_P3 | 173 | S. intermedius |
| | | | | 173 |
| JF496731 | Banklt1436913-14 | Seq165_P3 | 165 | S. milleri 165 |
| JF496732 | Banklt1436913-15 | Seq166_P3 | 166 | S. mitis 166 |
| JF496733 | Banklt1436913-16 | Seq185_P3 | 185 | S. mitis* 185 |
| JF496734 | Banklt1436913-17 | Seq180B_P3 | 180B | S. oralis 180B |
| JF496735 | Banklt1436913-18 | Seq179_P3 | 179 | S. sanguinis |
| | | | | 179 |
| JF496736 | Banklt1436913-19 | Seq103_P3 | 103 | V. dispar 103 |

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of treating periodontitis, comprising:
administering a pharmaceutical composition comprising one or more distinct bacteria selected from the group consisting of strains of Actinomyces naeslundii, Propionibacterium acnes, Actinomyces odontolyticus, Streptococcus intermedius, Streptococcus milleri, Streptococcus mitis, Streptococcus oxalis, Streptococcus sanguinis and Veillonella dispar to a subject exhibiting symptoms of periodontitis.

2. The method of claim 1, wherein said administering reduces symptoms of periodontitis in said subject.

3. The method of claim 1, wherein bacteria is selected from the group consisting of Actinomyces naeslundii 106, Actinomyces naeslundii 109A, Actinomyces naeslundii 110A, Actinomyces naeslundii 139A, Actinomyces naeslundii 141, Actinomyces naeslundii 144A, Actinomyces naeslundii 164B, Actinomyces naeslundii 181A, Propionibacterium acnes 115, Propionibacterium acnes 167, Propionibacterium acnes 168, Actinomyces odontolyticus 178, Streptococcus intermedius 173, Streptococcus milleri 165, Streptococcus mitis 166, Streptococcus mitis 185, Streptococcus oxalis 180B, Streptococcus sanguinis 179 and Veillonella dispar 103.

4. The method of claim 3, wherein said bacteria is selected from the group consisting of Actinomyces naeslundii 106, Actinomyces naeslundii 139A, Actinomyces naeslundii 144A, Actinomyces naeslundii 164B, Streptococcus intermedius 173 and Streptococcus oxalis 180B.

5. The method of claim 1, wherein said administering inhibits the growth of Porphyromonas gingivalis in said subject's mouth.

6. The method of claim 1, wherein said administering comprises topical administration to the mouth of said subject.

7. The method of claim 1, wherein said composition further comprises one or more anti-periodontitis components.

8. A method of preventing periodontitis, comprising:
administering a pharmaceutical composition comprising one or more distinct bacteria selected from the group consisting of strains of Actinomyces naeslundii, Propionibacterium acnes, Actinomyces odontolyticus, Streptococcus intermedius, Streptococcus milleri, Streptococcus mitis, Streptococcus oxalis, Streptococcus sanguinis and Veillonella dispar to a subject at risk of exhibiting symptoms of periodontitis;
wherein said administering prevents the development of symptoms of periodontitis in said subject.

9. The method of claim 8, wherein bacteria is selected from the group consisting of Actinomyces naeslundii 106, Actinomyces naeslundii 109A, Actinomyces naeslundii 110A, Actinomyces naeslundii 139A, Actinomyces naeslundii 141, Actinomyces naeslundii 144A, Actinomyces naeslundii 164B, Actinomyces naeslundii 181A, Propionibacterium acnes 115, Propionibacterium acnes 167, Propionibacterium acnes 168, Actinomyces odontolyticus 178, Streptococcus intermedius 173, Streptococcus milleri 165, Streptococcus mitis 166, Streptococcus mitis 185, Streptococcus oxalis 180B, Streptococcus sanguinis 179 and Veillonella dispar 103.

10. The method of claim 9, wherein said bacteria is selected from the group consisting of Actinomyces naeslundii 106, Actinomyces naeslundii 139A, Actinomyces naeslundii 144A, Actinomyces naeslundii 164B, Streptococcus intermedius 173 and Streptococcus oxalis 180B.

11. The method of claim 8, wherein said administering inhibits the growth of Porphyromonas gingivalis in said subject's mouth.

12. The method of claim 8, wherein said administering comprises topical administration to the mouth of said subject.

13. The method of claim 8, wherein said composition further comprises one or more anti-periodontitis components.

14. A pharmaceutical composition, comprising:
a) one or more distinct bacteria selected from the group consisting of strains of Actinomyces naeslundii, Propionibacterium acnes, Actinomyces odontolyticus, Streptococcus intermedius, Streptococcus milleri, Streptococcus mitis, Streptococcus oxalis, Streptococcus sanguinis and Veillonella dispar; and
b) a pharmaceutically acceptable carrier.

15. The composition of claim 14, wherein bacteria is selected from the group consisting of Actinomyces naeslundii 106, Actinomyces naeslundii 109A, Actinomyces naeslundii 110A, Actinomyces naeslundii 139A, Actinomyces naeslundii 141, Actinomyces naeslundii 144A, Actinomyces naeslundii 164B, Actinomyces naeslundii 181A, Propionibacterium acnes 115, Propionibacterium acnes 167, Propionibacterium acnes 168, Actinomyces odontolyticus 178, Streptococcus intermedius 173, Streptococcus milleri 165, Streptococcus mitis 166, Streptococcus mitis 185, Streptococcus oxalis 180B, Streptococcus sanguinis 179 and Veillonella dispar 103.

16. The composition of claim 15, wherein said bacteria is selected from the group consisting of Actinomyces naeslundii 106, Actinomyces naeslundii 139A, Actinomyces naeslundii 144A, Actinomyces naeslundii 164B, Streptococcus intermedius 173 and Streptococcus oxalis 180B.

17. The composition of claim 14, wherein said composition further comprises one or more anti-periodontitis components.

18. The composition of claim 14, wherein said composition further comprises one or more flavorings and/or calorie free sweeteners.

19. The composition of claim 14, wherein said composition is adapted for oral topical administration.
